(19) United States
(12) Patent Application Publication
(10) Pub. No.: US 2010/0273326 A1
(43) Pub. Date: Oct. 28, 2010

(54) METHOD FOR PURIFYING UNSATURATED FLUOROCARBON COMPOUND, METHOD FOR FORMING FLUOROCARBON FILM, AND METHOD FOR PRODUCING SEMICONDUCTOR DEVICE

(76) Inventors: Masahiro Nakamura, Tokyo (JP); Yuka Soma, Tokyo (JP)

Correspondence Address: EDWARDS ANGELL PALMER & DODGE LLP, P.O. BOX 55874, BOSTON, MA, 02205, US

(21) Appl. No.: 12/085,675

(22) PCT Filed: Nov. 30, 2006
(86) PCT No.: PCT/JP2006/323929

§ 371 Date: Jul. 6, 2010

(30) Foreign Application Priority Data
Nov. 30, 2005 (JP) 2005-345071

(51) Int. Cl. H01L 21/30; C07C 17/38; H05H 1/24

(52) U.S. Cl. 438/680; 570/177; 427/569; 257/E21.211

(57) ABSTRACT

A method for purifying an unsaturated fluorocarbon compound includes causing a crude unsaturated fluorocarbon compound shown by the formula C₅F₈ or C₄F₆ to come in contact with a boron oxide to obtain a purified unsaturated fluorocarbon compound. A method for forming a fluorocarbon film includes forming a fluorocarbon film by a CVD method using the purified unsaturated fluorocarbon compound as a plasma reaction gas, and a method for producing a semiconductor device includes a step of forming a fluorocarbon film by a CVD method. Because the purified unsaturated fluorocarbon compound obtained by the above method has a high purity and an extremely low water content, the compound may be suitably used as a plasma reaction gas for forming a fluorocarbon film using a plasma CVD method or a plasma reaction gas used for a semiconductor device production process including a fluorocarbon film formation step by a CVD method.

### BACKGROUND OF THE INVENTION

1. Field of the Invention

The present invention relates to a method for purifying an unsaturated fluorocarbon compound shown by the formula C₅F₈ or C₄F₆ which is useful for producing a semiconductor device, a method for forming a fluorocarbon film by chemical vapor deposition (CVD) using the purified unsaturated fluorocarbon compound obtained by the purification method as a plasma reaction gas, and a method for producing a semiconductor device.

2. Description of Related Art

An unsaturated fluorocarbon compound shown by the formula C₅F₈ or C₄F₆ has been widely used as a plasma reaction gas.

As a purification method for the unsaturated fluorocarbon compound, a method using a molecular sieve (crystalline aluminosilicates), which is a common dehydration agent, has been known.

However, when the unsaturated fluorocarbon compound comes in contact with the molecular sieve, an isomerization reaction, a decomposition reaction, or the like occurs easily, inducing a decrease in purity.

In order to solve this problem, Patent Document 1 proposes a method of purifying hexafluoro-1,3-butadiene using a molecular sieve with an average pore size of 5 Å. According to this method, high purity hexafluoro-1,3-butadiene can be obtained while suppressing isomerization into hexafluoro-2-butyne.

However, the method disclosed in this document may not be able to suppress the isomerization or may produce other decomposed materials depending on the kind of the unsaturated fluorocarbon compound to be purified.

Patent Document 2 proposes a method of purifying a perfluoro compound by treating the perfluoro compound with a molecular sieve after treating with activated carbon. This method can decrease impurities such as hydrogen fluoride (HF) and water existing in a perfluoro compound to 1 ppm or less.

However, this method is not industrially useful because the operation is complicated due to the requirement of two process steps, one a treatment with activated carbon, and the other a treatment with the molecular sieve.

Patent Document 3 proposes a method of purifying an unsaturated fluorocarbon compound by removing gas from the gaseous phase while applying pressure to the unsaturated fluorocarbon compound at 1.27×10⁵ Pa or more. The document also mentions that it is preferable to cause the unsaturated fluorocarbon compound to come in contact with a calcined metal oxide in addition to the gas removal operation.

However, the Patent Document 3 only describes a case of using aluminum oxide (Al₂O₃) (Examples 1 to 4).

Patent Document 1: U.S. Pat. No. 6,544,319

### Patent Document 2: JP-A-2004-339187

### Patent Document 3: JP-A-2005-239596

### SUMMARY OF THE INVENTION

As stated above, a variety of techniques have been proposed as a method for purifying the unsaturated fluorocarbon compound. However, the purity of the unsaturated fluorocarbon compound in a plasma reaction gas obtained by the known purification method is approximately 99.9 vol %, and the water content is approximately 1 ppm. Along with the rapid progress of semiconductor devices, a technology for forming more uniform fluorocarbon films with higher quality has been desired in the manufacture of semiconductor devices in recent years. A plasma reaction gas used in the manufacture of semiconductor devices is required to have high purity.

The present invention has been achieved in view of this situation in general technology and has an object of providing a method for purifying an unsaturated fluorocarbon compound shown by the formula C₅F₈ or C₄F₆, which can produce an unsaturated fluorocarbon compound with a purity of 99.999 vol % or more and a water content of 500 ppb by volume or less, for example, a method for forming a fluorocarbon film by the CVD method in which the unsaturated fluorocarbon compound purified by the above method is used as a plasma reaction gas, and a method for producing a semiconductor device.

As a result of extensive studies in order to achieve the above object, the inventors of the present invention have found that a purified unsaturated fluorocarbon compound with a purity of 99.999 vol % or more and a water content of 500 ppb by volume or less can be produced without inducing an isomerization reaction or decomposition reaction by causing a crude unsaturated fluorocarbon compound shown by the formula C₅F₈ or C₄F₆ to come in contact with a boron oxide. The inventors of the present invention have also found that the high purity unsaturated fluorocarbon compound purified by this method is useful as a plasma reaction gas for forming a fluorocarbon film by the CVD method. These findings have led to the completion of the present invention.

A first aspect of the present invention provides a method for purifying an unsaturated fluorocarbon compound comprising causing a crude unsaturated fluorocarbon compound shown by the formula C₅F₈ or C₄F₆ to come in contact with a boron oxide to produce a purified unsaturated fluorocarbon compound.

In the purification method of the present invention, the unsaturated fluorocarbon compound is preferably octafluoro-2-pentyne, octafluorocyclopentene, hexafluoro-2-butyne, or hexafluoro-1,3-butadiene.

The purification method of the present invention preferably removes water contained as impurities. More preferably, the purified unsaturated fluorocarbon compound has a purity of 99.999 vol % or more and a water content of 500 ppb by volume or less.

A second aspect of the present invention provides a method for forming a fluorocarbon film comprising forming a fluorocarbon film by a CVD method using a purified unsaturated fluorocarbon compound obtained by the purification method of the present invention as a plasma reaction gas.

A third aspect of the present invention provides a method for producing a semiconductor device comprising a step of forming a fluorocarbon film by a CVD method using a purified unsaturated fluorocarbon compound obtained by the purification method of the present invention as a plasma reaction gas.

The purification method of the present invention is capable of removing impurities without inducing isomerization and decomposition reaction. A purified unsaturated fluorocarbon compound with a purity of 99.999 vol % or more and a water content of 500 ppb by volume or less, for example, can be obtained.

Because the purified unsaturated fluorocarbon compound purified by the method of the present invention has a high purity and an extremely low water content, the purified compound is particularly useful as a plasma reaction gas for forming fluorocarbon films using a plasma CVD method and a plasma reaction gas during production of a semiconductor device comprising a step of fluorocarbon film formation by the CVD method.

The method for forming a fluorocarbon film of the present invention is capable of preventing generation of water-derived corrosive gas and decrease of adhesion because the method uses the purified unsaturated fluorocarbon compound obtained by the method of the present invention as a plasma reaction gas. Therefore, the method is capable of forming a uniform interlayer dielectric film (fluorocarbon film) with high quality and good reproducibility.

The method for producing a semiconductor device of the present invention is capable of efficiently producing a highly densified, high performance semiconductor device on a large-caliber wafer by using the purified unsaturated fluorocarbon compound obtained by the method of the present invention as a plasma reaction gas in the fluorocarbon film formation by the CVD method.

### DETAILED DESCRIPTION OF THE INVENTION AND PREFERRED EMBODIMENTS

The present invention will be described in detail below.

### 1) Method for Purifying Unsaturated Fluorocarbon Compound

The purification method of the present invention comprises causing a crude unsaturated fluorocarbon compound shown by the formula C₅F₈ or C₄F₆ to come in contact with a boron oxide.

The unsaturated fluorocarbon compound shown by the formula C₅F₈ or C₄F₆ (hereinafter abbreviated as “unsaturated fluorocarbon compound”) can be used without particular limitations as long as the compound can be shown by the formula C₅F₈ or C₄F₆.

As specific examples, the unsaturated fluorocarbon compound shown by the formula C₅F₈ such as octafluoro-1-pentyne, octafluoro-2-pentyne, octafluoro-1,3-pentadiene, octafluoro-1,4-pentadiene, octafluorocyclopentene, octafluoroisoprene, octafluoro-(1-methylcyclobutene), and octafluoro-(1,2-dimethylcyclopropene); and the unsaturated fluorocarbon compound shown by the formula C₄F₆ such as hexafluoro-2-butyne, hexafluoro-1-butyne, hexafluorocyclobutene, hexafluoro-1,3-butadiene, and hexafluoro-(1-methylcyclopropene) can be given.

Of these, octafluoro-2-pentyne, octafluorocyclopentene, hexafluoro-2-butyne and hexafluoro-1,3-butadiene are preferable because of their industrial usefulness. Octafluoro-2-pentyne is particularly preferable.

These unsaturated fluorocarbon compounds are known compounds. The term “crude unsaturated fluorocarbon compound” in the present invention refers to a compound to be purified by contact with a boron oxide. The crude compounds described below are usually used in the present invention as is, or the compound may be purified by a purification method (including the purification method of the present invention) before being purified by contact with a boron oxide.

The crude unsaturated fluorocarbon compound of the invention can be prepared by a known method. For example, crude octafluoro-2-pentyne may be prepared by the method disclosed in JP-A-2003-146917, crude octafluorocyclopenetene may be prepared by the method disclosed in JP-A-2005-239596, and crude hexafluoro-1,3-butadiene and crude hexafluoro-2-butyne may be prepared by the method disclosed in US 200-5247670. It is also possible to use these unsaturated fluorocarbon compounds, which are commercially available, as the crude unsaturated fluorocarbon compounds in the present invention.

As examples of the boron oxides used in the present invention, diboron dioxide, diboron trioxide, tetraboron trioxide, tetraboron pentaoxide, and the like can be given. Of these, diboron trioxide is particularly preferable because dehydration can be carried out efficiently without an isomerization reaction and a decomposition reaction when causing an unsaturated fluorocarbon compound to come in contact with diboron trioxide. The boron oxide used may be prepared by a known method or a commercially-available boron oxide may be used.

The boron oxides are used in an amount of usually 1 to 50 parts by weight, and preferably 5 to 30 parts by weight for 100 parts by weight of the unsaturated fluorocarbon compound.

The boron oxides used within this range may sufficiently purify the unsaturated fluorocarbon compound. Using an excessive amount of boron oxides is not preferable because not only is no more purifying effect expected, but also the purifying cost increases by the use of such an excessive amount of boron oxides.

It is preferable that the boron oxide be activated before use in order to increase its purifying capability.

As examples of the activation treatment of the boron oxide, (i) a method of heating under reduced pressure and (ii) a method of heating in an inert gas stream such as nitrogen or argon can be given.

The amount of impurities such as water and oxygen contained in the inert gas used in the method (ii) is 100 ppb by volume or less, preferably 10 ppb by volume or less, and more preferably 1 ppb by volume or less.

The temperature for the heat treatment is usually 100° C. or more, and preferably 120° C. or more in the methods (i) and (ii).

The activation treatment of the boron oxide is preferably performed after filling the boron oxide in the below-described purification container to prevent contamination.

As a method of causing the crude unsaturated fluorocarbon compound to come in contact with the boron oxide, (a) an immersion method of adding the crude unsaturated fluorocarbon compound to be purified to the boron oxide in a container and allowing the mixture to stand and (b) a circulation method of causing the gaseous crude unsaturated fluorocarbon compound to flow in a pipe filled with the boron oxide and causing mutual contact can be given. The method (b) is preferable since the purification can be efficiently carried out continuously.

As an example of an apparatus to carry out the method (b), a sealing container to seal in the crude unsaturated fluorocarbon compound, a massflow controller to control the flow rate of the crude unsaturated fluorocarbon compound, a purification container with the boron oxide contained therein, and a container to collect the purified unsaturated fluorocarbon compound, all connected in that order, can be given.

The unsaturated fluorocarbon compound is purified using this apparatus by the following method.

First, the crude unsaturated fluorocarbon compound sealed in the sealing container is caused to flow into the purification container filled with the boron oxides while controlling the flow rate by the massflow controller. Impurities (e.g. very small amount of water) contained in the crude unsaturated fluorocarbon compound is efficiently removed by causing the gaseous crude unsaturated fluorocarbon compound to come in contact with the boron oxide. In this instance, isomerization and decomposition reactions that occur when a molecular sieve is used do not occur. Subsequently, the purified unsaturated fluorocarbon compound is collected in the container.

It is preferable to previously discharge air from the purification system consisting of the sealing container to seal in the crude unsaturated fluorocarbon compound to be purified, the purification container, and the collecting container using a vacuum pump in order to prevent the purified compound from being contaminated by water and the like.

It is also preferable to sufficiently cool the collecting container before starting the purifying operation. The cooling temperature is below the boiling point of the unsaturated fluorocarbon compound to be used. From the viewpoint of efficient collection, the temperature is preferably 10° C. or more lower than the boiling point, and more preferably 50° C. or more lower than the boiling point of the compound.

In the method (a) or (b), the conditions such as temperature, pressure, and flow rate when causing the crude unsaturated fluorocarbon compound to come in contact with the boron oxide are appropriately selected according to the kind of the unsaturated fluorocarbon compound to be used.

The temperature when causing the unsaturated fluorocarbon compound to come in contact with the boron oxide is usually 120° C. or less, preferably 80° C. or less, and more preferably 10 to 50° C. in order to achieve sufficient purifying capability.

In the method (a) or (b), the pressure when causing the unsaturated fluorocarbon compound to come in contact with the boron oxide is usually 0.01 to 1 MPa, preferably 0.02 to 0.3 MPa, and more preferably 0.04 to 0.1 MPa, in terms of absolute pressure.

In the method (b), the flow rate of the crude unsaturated fluorocarbon compound is selected from between 10 ml/min and 60 l/min according to the size of the purification container. The flow rate of the crude compound is usually 10 ml/min to 1 l/min.

According to the purification method of the present invention, a purified unsaturated fluorocarbon compound with an extremely high purity and an extremely low water content can be obtained.

The purity of the purified unsaturated fluorocarbon compound is usually 99.999 vol % or more, and the water content is usually 500 ppb by volume or less, preferably 100 ppb by volume or less, and particularly preferably 50 ppb by volume or less. The purity and the water content of the unsaturated fluorocarbon compound cannot be measured simultaneously. The purity is measured by a gas chromatography analysis using a flame ionization detector (FID) and the water content is measured using a highly sensitive water analyzer.

The purified unsaturated fluorocarbon compound purified by the purification method of the present invention is suitably used in the fields of semiconductor device manufacturing, electronics and electricity, precision machining, and the like.

Because the purified unsaturated fluorocarbon compound purified by the method of the present invention has a high purity and an extremely low water content, the compound is particularly useful as a plasma reaction gas for forming a fluorocarbon film using the plasma CVD method and a plasma reaction gas during production of a semiconductor device comprising fluorocarbon film formation by the CVD method.

### 2) Method for Forming Fluorocarbon Film

The method for forming fluorocarbon films by the CVD method comprises using the purified unsaturated fluorocarbon compound obtained by the purification method of the present invention as a plasma reaction gas. Since the purified unsaturated fluorocarbon compound obtained by the purification method of the present invention has an extremely low water content, generation of water-derived corrosive gas and decrease of adhesion do not occur. In addition, because the compound has an extremely high purity, a uniform interlayer dielectric film (fluorocarbon film) can be formed with good reproducibility.

In the method for forming fluorocarbon films by using a plasma reaction gas, a thin fluorocarbon film is formed on various surfaces to be treated by activating and polymerizing the unsaturated fluorocarbon compound by plasma discharge.

As the CVD method using plasma, general methods such as the method disclosed in JP-A-9-237783 may be used. Plasma is usually generated under the conditions of a high frequency (RF) output of 10 W to 10 kW, a target material temperature of 0 to 500° C., and a reaction chamber pressure of 0.005 to 13.3 kPa. A high plasma density, usually of 10¹⁰ cm⁻³ or more, and particularly 10¹⁰ to 10¹² cm⁻³ is preferable.

A parallel plate CVD device is generally used for the plasma CVD. A microwave CVD device, an ECR-CVD device, and a high-density plasma CVD device (helicon wave type or high frequency induction type) may also be used.

In the present invention, it is preferable to connect the apparatus for purifying the unsaturated fluorocarbon compound to the plasma CVD device so that the purified unsaturated fluorocarbon compound is directly introduced into the plasma generating chamber.

An inert gas such as helium, neon, and argon may be added to the purified unsaturated fluorocarbon compound to be used in order to control concentration of activated species generating in plasma and accelerate dissociation of the raw material gas. These inert gases may be used either individually or in combination of two or more.

The volume ratio of the total inert gas to the purified unsaturated fluorocarbon compound (inert gas/purified unsaturated fluorocarbon compound) is preferably 2 to 200, and particularly preferably 5 to 150.

There are no particular limitations to the object to be treated. For examples, surfaces of articles or parts which require functions or properties such as insulation, water repellency, corrosion resistance, acid resistance, lubricity, or antireflection in the fields of semiconductor manufacturing, electronics and electricity, precision machining, and the like can be given. The films are preferably formed on the surfaces of articles and parts, particularly substrates, which require insulation in the fields of semiconductor manufacturing and electronics and electricity.

### 3) Method for Producing Semiconductor Device

The method for producing a semiconductor device comprises a step of forming the fluorocarbon film by the CVD method using the purified unsaturated fluorocarbon compound obtained by the purification method of the present invention as a gas (raw material gas) for plasma reaction.

In the method for producing semiconductor device of the present invention, the same method of forming fluorocarbon film of the present invention may be applied to the step of forming the fluorocarbon film by the CVD method. A semiconductor device may be produced according to the general methods such as the method described in U.S. Pat. No. 5,242,852.

The method for producing a semiconductor device of the present invention is capable of efficiently producing a high quality and high performance semiconductor device by using the purified unsaturated fluorocarbon compound having a high purity and an extremely low water content obtained by the purification method of the present invention as a plasma reaction gas.

### EXAMPLES

The present invention will be described in more detail by way of examples. Note that the present invention is not limited to the following examples. The analysis of purity and water content was conducted by the following methods.

### (1) Analysis of Purity

The purity of the crude and the purified unsaturated fluorocarbon compound was analyzed using gas chromatography under the following conditions.

Equipment: “HP6890” manufactured by Hewlett-Packard Company
Column: “Ultra Alloy+−1(s)” (length: 60 m, inner diameter 0.25 mm, film thickness: 0.4 μm) manufactured by Frontier Laboratories Ltd.
Column temperature: maintained at −20° C. for 15 minutes, then increased to 200° C. at a rate of 20° C./min
Injection temperature: 200° C.
Carrier gas: nitrogen gas (flow rate: 1 ml/min)

### Detector: FID

Internal standard substance: n-butane

### (2) Measurement of Water Content

The water content of the crude and the purified unsaturated fluorocarbon compound was measured by cavity ring-down spectroscopy using a high sensitivity water content analyzer under the following conditions.

Measuring device: “Laser Trace” manufactured by Tiger Optics
Detection limit: 5 ppb by volume

### Example 1

The following experiment was carried out using a purifying system having a sealing container to seal in the crude unsaturated fluorocarbon compounds, a massflow controller to control the flow rate of the crude unsaturated fluorocarbon compounds, a purification container with a diameter of 38 mm and a length of 40 mm filled with 40 g of boron oxide (B₂O₃), and a container to collect the purified unsaturated fluorocarbon compounds. The collecting container was previously cooled to −78° C.

Octafluoro-2-pentyne having a purity of 99.950 vol % and a water content of 15 ppm by volume was packed in a sealing container. The container was connected to the purification container filled with the boron oxide. The crude octafluoro-2-pentyne in the sealing container was fed to the purification container and purified (caused to come in contact with boron oxide) while controlling the inlet pressure of the purification container at 0.1 MPa, the temperature at 23° C., and the flow rate at 100 ml/min. The purified octafluoro-2-pentyne was collected in the collecting container. The purity and the water content of the purified octafluoro-2-pentyne collected were measured. The purity was 99.999 vol % or more and the water content was 50 ppb by volume.

### Example 2

The following experiment was carried out using the same purifying system as in Example 1.

Octafluorocyclopentene having a purity of 99.980 vol % and a water content of 10 ppm by volume was packed in a sealing container. The container was connected to the purification container filled with the boron oxide. The crude octafluorocyclopentene in the sealing container was fed to the purification container and purified while controlling the inlet pressure of the purification container at 0.04 MPa, the temperature at 23° C., and the flow rate at 100 ml/min. The purified octafluorocyclopentene was collected in the collecting container. The purity and the water content of the purified octafluorocyclopentene collected were measured. The purity was 99.999 vol % or more and the water content was 35 ppb by volume.

### Example 3

The following experiment was carried out using the same purifying system as in Example 1.

Octafluoro-2-pentyne having a purity of 99.950 vol % and a water content of 80 ppm by volume was packed in a sealing container. The container was connected to the purification container filled with the boron oxide. The crude octafluoro-2-pentyne in the sealing container was fed to the purification container and purified while controlling the inlet pressure of the purification container at 0.1 MPa, the temperature at 23° C., and the flow rate at 100 ml/min. The purified octafluoro-2-pentyne was collected in the collecting container. The purity and the water content of the purified octafluoro-2-pentyne collected were measured. The purity was 99.999 vol % or more and the water content was 50 ppb by volume.

### Comparative Example 1

Crude octafluoro-2-pentyne was purified in the same manner as in Example 1 except for using 32 g of molecular sieves (MS-3A) instead of the boron oxide to fill in the purification container. The purity and the water content of the octafluoro-2-pentyne in the purified octafluoro-2-pentyne were measured. The purity was 99.822 vol % and the water content was 82 ppb by volume.

### Comparative Example 2

Crude octafluoro-2-pentyne was purified in the same manner as in Example 1 except for using 37 g of alumina (Al₂O₃) instead of the boron oxide in the purification container. The purity and the water content of the octafluoro-2-pentyne in the purified octafluoro-2-pentyne were measured. The purity was 99.901 vol % and the water content was 362 ppb by volume.

1. A method for purifying an unsaturated fluorocarbon compound comprising causing a crude unsaturated fluorocarbon compound shown by the formula C₅F₈ or C₄F₆ to come in contact with a boron oxide to produce a purified unsaturated fluorocarbon compound.

2. The method according to claim 1, wherein the unsaturated fluorinated carbon compound is octafluoro-2-pentyne, octafluorocyclopentene, hexafluoro-2-butyne, or hexafluoro-1,3-butadiene.

3. The method according to claim 1, comprising removing water contained as impurities.

4. The method according to claim 1, wherein the purified unsaturated fluorocarbon compound has a purity of 99.999 vol % or more and a water content of 500 ppb by volume or less.

5. A method for forming a fluorocarbon film comprising forming a fluorocarbon film by a CVD method using a purified unsaturated fluorocarbon compound obtained by the method according to claim 1 as a plasma reaction gas.

6. A method for producing a semiconductor device comprising a step of forming a fluorocarbon film by a CVD method using a purified unsaturated fluorocarbon compound obtained by the method according to claim 1 as a plasma reaction gas.

7. The method according to claim 2, comprising removing water contained as impurities.

8. The method according to claim 2, wherein the purified unsaturated fluorocarbon compound has a purity of 99.999 vol % or more and a water content of 500 ppb by volume or less.

9. The method according to claim 3, wherein the purified unsaturated fluorocarbon compound has a purity of 99.999 vol % or more and a water content of 500 ppb by volume or less.

10. The method according to claim 7, wherein the purified unsaturated fluorocarbon compound has a purity of 99.999 vol % or more and a water content of 500 ppb by volume or less.

11. A method for forming a fluorocarbon film comprising forming a fluorocarbon film by a CVD method using a purified unsaturated fluorocarbon compound obtained by the method according to claim 2 as a plasma reaction gas.

12. A method for forming a fluorocarbon film comprising forming a fluorocarbon film by a CVD method using a purified unsaturated fluorocarbon compound obtained by the method according to claim 3 as a plasma reaction gas.

13. A method for forming a fluorocarbon film comprising forming a fluorocarbon film by a CVD method using a purified unsaturated fluorocarbon compound obtained by the method according to claim 4 as a plasma reaction gas.

14. A method for forming a fluorocarbon film comprising forming a fluorocarbon film by a CVD method using a purified unsaturated fluorocarbon compound obtained by the method according to claim 7 as a plasma reaction gas.

15. A method for producing a semiconductor device comprising a step of forming a fluorocarbon film by a CVD method using a purified unsaturated fluorocarbon compound obtained by the method according to claim 2 as a plasma reaction gas.

16. A method for producing a semiconductor device comprising a step of forming a fluorocarbon film by a CVD method using a purified unsaturated fluorocarbon compound obtained by the method according to claim 3 as a plasma reaction gas.

17. A method for producing a semiconductor device comprising a step of forming a fluorocarbon film by a CVD method using a purified unsaturated fluorocarbon compound obtained by the method according to claim 4 as a plasma reaction gas.

18. A method for producing a semiconductor device comprising a step of forming a fluorocarbon film by a CVD method using a purified unsaturated fluorocarbon compound obtained by the method according to claim 5 as a plasma reaction gas.

19. A method for producing a semiconductor device comprising a step of forming a fluorocarbon film by a CVD method using a purified unsaturated fluorocarbon compound obtained by the method according to claim 7 as a plasma reaction gas.

20. A method for producing a semiconductor device comprising a step of forming a fluorocarbon film by a CVD method using a purified unsaturated fluorocarbon compound obtained by the method according to claim 8 as a plasma reaction gas.
